(19) United States
(12) Patent Application Publication
(10) Pub. No.: US 2013/0245546 A1
(43) Pub. Date: Sep. 19, 2013

(54) FUNCTIONAL ADHESIVE BANDAGE WITH SENSOR AND ACTUATOR

(71) Applicant: ROBERT BOSCH GMBH, Stuttgart (DE)

(72) Inventor: Henning Hayn, Stuttgart (DE)

(73) Assignee: Robert Bosch GmbH, Stuttgart (DE)

(21) Appl. No.: 13/796,992

(22) Filed: Mar. 12, 2013

(30) Foreign Application Priority Data
Mar. 13, 2012 (DE) 10 2012 203 880.9

(51) Int. Cl. A61M 5/172; A61F 7/00; A61N 1/36; A61N 5/06

(52) U.S. Cl. CPC A61M 5/1723; A61N 5/0624; A61F 7/00; A61N 1/36
USPC 604/66; 607/88; 607/96; 607/2

(57) ABSTRACT

A functional adhesive bandage includes an adhesive layer and a cover layer. A sensor for measuring a vital parameter, a control unit, an energy storage device, and an actuator are arranged between the adhesive layer and the cover layer. The control unit is configured to evaluate the vital parameter measured by the sensor. The control unit is further configured to regulate the actuator based on the evaluation.

This application claims priority under 37 U.S.C. §119 to patent application no. DE 10 2012 203 880.9, filed on Mar. 13, 2012 in Germany, the disclosure of which is incorporated herein by reference in its entirety.

### BACKGROUND

The disclosure proceeds from a functional adhesive bandage and a method according to the description below.

A functional adhesive bandage is an adhesive bandage which has electronic components for monitoring human vital parameters. By way of example, the components comprise sensors, microprocessors, display and operating element. By way of example, the sensors of the functional adhesive bandage are used to measure vital data, such as e.g. blood oxygen saturation, EKG, EMG. By way of example, the vital data is then stored, evaluated or transmitted to an evaluation unit.

DE102009029215 has already disclosed a functional adhesive bandage for acoustic detection of sleep apnea.

US20050226921 and U.S. Pat. No. 5,879,322 describe devices for transdermal release of active ingredients. The devices are applied to skin and the release of active ingredients is controlled electronically.

### SUMMARY

A functional adhesive bandage according to the disclosure has an adhesive layer and a cover layer. Here, a sensor for measuring a vital parameter, a control unit, an energy storage device and an actuator are arranged between the adhesive layer and the cover layer. The control unit is configured to evaluate the vital parameters measured by the sensor and to regulate the actuator on the basis of the evaluation. The functional adhesive bandage according to the disclosure is advantageous because it has a closed sensor/actuator regulation by means of which vital parameters can be set.

The measures listed in the description below render possible advantageous developments and improvements of the functional adhesive bandage specified below.

A functional adhesive bandage is advantageous if the actuator is an electromagnetic radiation source. By way of example, the electromagnetic radiation source is used to disinfect a skin surface or a wound, above which the functional adhesive bandage is applied. By way of example, an ultraviolet lamp is used as electromagnetic radiation source.

A functional adhesive bandage is particularly advantageous if the actuator is a delivery unit by means of which an active ingredient can be applied from a reservoir onto the skin, a wound, or under the skin and into the body. By way of example, the delivery unit is an electric pump and the vital parameter that can be set is the pH value of a wound.

The actuator is expediently embodied as a heating- and/or a cooling element, which regulates the temperature under the functional adhesive bandage arranged on a skin surface or body part.

It is advantageous if the actuator is embodied as an electrode. This is because the functional adhesive bandage is then, when applied onto a body surface, capable of activating a stimulation of muscles or promoting the perfusion of skin surfaces by means of the electrode.

Furthermore, it is advantageous if the actuator is embodied as mechanical, pneumatic or hydraulic actuator, by means of which a movement of a body part is supported.

It is furthermore advantageous if the actuator of the functional adhesive bandage is a converter of mechanical and/or thermal energy into electrical energy. By way of example, as a result of this energy is obtained by movement of the functional adhesive bandage or by warmth surrounding the functional adhesive bandage. The energy obtained is, for example, fed to the energy storage device during this so-called energy harvesting. Thus, this renders possible an increase of the service life of the energy storage device and/or an independent energy supply of the functional adhesive bandage.

A method according to the disclosure for regulating a vital parameter by means of a functional adhesive bandage comprises the following steps: prescribing an intended variable for the vital parameter, measuring an actual variable for the vital parameter, establishing a deviation of the actual variable from the intended variable, setting a control variable on the basis of the deviation. The method according to the disclosure has the advantage of regulating a vital parameter to the predetermined intended variable by setting the control variable.

### BRIEF DESCRIPTION OF THE DRAWINGS

In detail:

FIG. 1 shows a schematic illustration of a first embodiment of a functional adhesive bandage according to the disclosure,

FIG. 2 shows a schematic illustration of a second embodiment of a functional adhesive bandage according to the disclosure,

FIG. 3 shows a block diagram of a method according to the disclosure, and

FIG. 4 shows a regulation of a vital variable using a functional adhesive bandage according to the disclosure.

### DETAILED DESCRIPTION

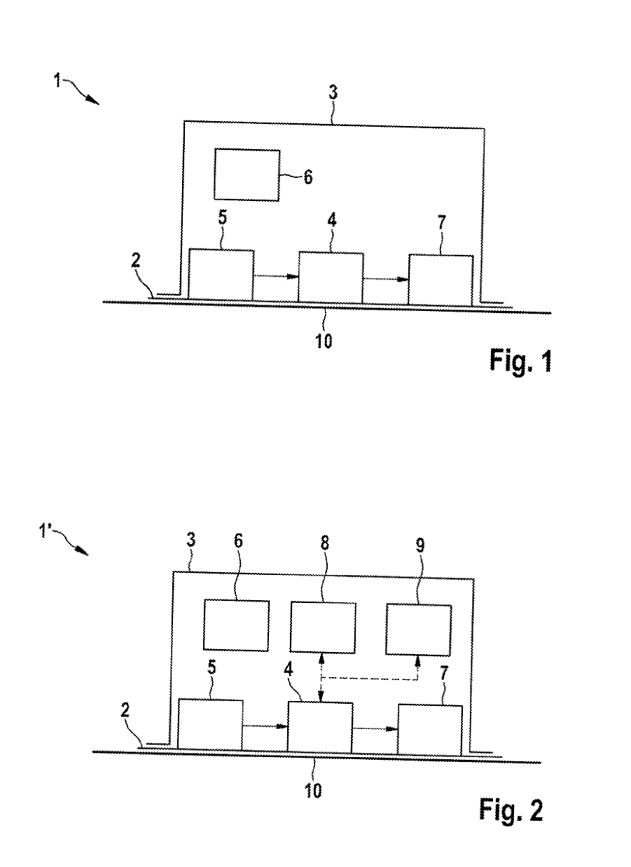

FIG. 1 shows a first embodiment of a functional adhesive bandage 1 according to the disclosure. The functional adhesive bandage is affixed to skin 10. The functional adhesive bandage 1 has an adhesive layer 2, a cover layer 3, a control unit 4, a sensor 5, an energy storage device 6 and an actuator 7. The control unit 4, the sensor 5, the energy storage device 6 and the actuator 7 are arranged between the adhesive layer 2 and the cover layer 3. The control unit 4, the sensor 5 and the actuator 7 are interconnected via data lines.

By way of example, the energy storage device 6 is a battery and/or a rechargeable battery. The energy storage device 6 supplies the other components, such as the control unit 4, the sensor 5 and the actuator 7, with energy via lines.

The sensor 5 is configured to measure vital parameters, for example oxygen saturation, blood sugar, pH value, temperature, moisture, position, acceleration and/or movement.

By way of example, the actuator 7 is an electrode, a hydraulic actuator, a pneumatic actuator, an electric motor, an electromagnetic radiation source, a heating- and/or a cooling element.

By way of example, the control unit 4 is a microcontroller with a storage device, which evaluates vital parameters or data measured by means of the sensor 5 and, on the basis of the evaluation, controls the actuator 7.

A second embodiment is shown in FIG. 2. The second embodiment of the functional adhesive bandage 1′ also has, compared to the first embodiment, a data transmission unit 8 and an operating unit 9 as further elements. The data transmission unit is arranged between the adhesive layer 2 and the cover layer 3. By way of example, the data transmission unit 8 is a module for transmitting data between the functional adhesive bandage 1′ and a base station, wherein the transmission takes place via e.g. WLAN, Bluetooth, ZigBee, GSM, 3G and/or 4G.

The operating unit 9 is arranged between the adhesive layer 2 and the cover layer 3 in such a way that a user can set an intended variable. By way of example, the operating unit 9 has a display, a pushbutton, a switch, a touchscreen, a loudspeaker and/or a microphone.

In a further embodiment, the actuator 7 is a converter of mechanical and/or thermal energy into electrical energy. The converter generates electrical energy from mechanical energy by a movement of the functional adhesive bandage 1, 1′ or from the heat of the surroundings. The generated energy is fed to the energy storage device 6 and/or other components of the functional adhesive bandage 1, 1′.

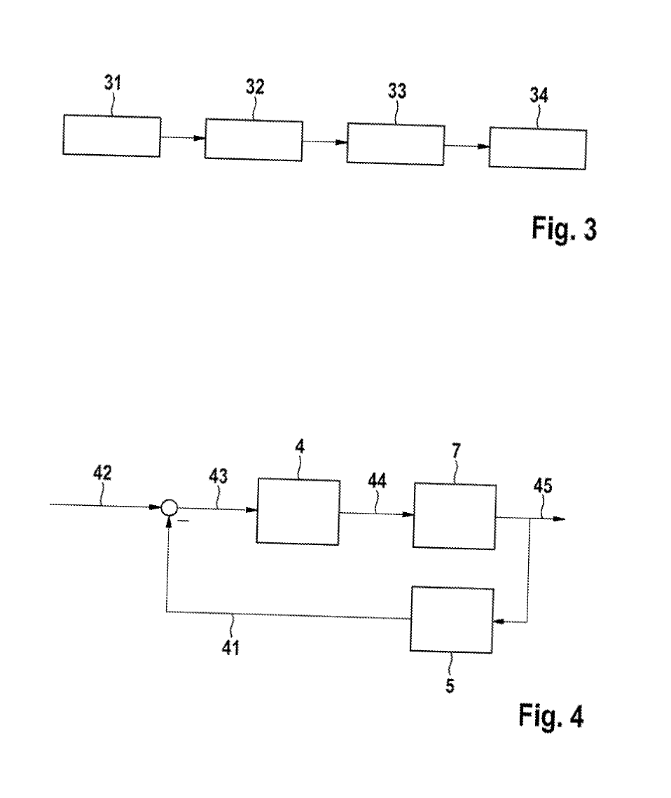

FIG. 3 shows the method for regulating a vital parameter. In a first method step 31, an intended variable for the vital parameter is set by the user. In a second method step 32, an actual variable for the vital parameter is measured. In a third method step 33, a deviation between measured actual variable and set intended variable is established. This deviation is used in a fourth method step 34 for setting a control variable. The control variable is set in a manner such that it has a retroactive effect on the vital parameter.

FIG. 4 shows how the control unit 4 with the sensor 4 and the actuator 7 regulates a vital parameter 45 as per the method shown in FIG. 2. A regulating error 43 is established from an actual variable 41 measured by the sensor 4 and an intended variable 42. The regulating error 43 is used by the control unit 4 for setting a control variable 44 of the actuator 7 in order to regulate the vital parameter 45 so that the vital parameter 45 follows the set intended variable 42.

What is claimed is:

1. A functional adhesive bandage, comprising:
an adhesive layer;
a cover layer;
a sensor configured to measure a vital parameter;
a control unit;
an energy storage device; and
an actuator; wherein:
the sensor, the control unit, the energy storage device, and the actuator are arranged between the adhesive layer and the cover layer,
the control unit is configured to evaluate the vital parameter measured by the sensor, and
the control unit is configured to regulate the actuator based on the evaluation.

2. The functional adhesive bandage according to claim 1, wherein the actuator is an electromagnetic radiation source.

3. The functional adhesive bandage according to claim 1, wherein the actuator is a delivery unit for an active ingredient.

4. The functional adhesive bandage according to claim 1, wherein the actuator is at least one of a heating element and a cooling element.

5. The functional adhesive bandage according to claim 1, wherein the actuator is an electrode.

6. The functional adhesive bandage according to claim 1, wherein the actuator is one of a mechanical actuator, a pneumatic actuator and a hydraulic actuator.

7. The functional adhesive bandage according to claim 1, wherein the actuator is a converter of at least one of mechanical energy and thermal energy into electrical energy.

8. A method for regulating a vital parameter with a functional adhesive bandage, comprising:
prescribing an intended variable for the vital parameter;
measuring an actual variable for the vital parameter;
establishing a deviation of the actual variable from the intended variable; and
setting a control variable based on the deviation.
